(19) United States
(12) Patent Application Publication
(10) Pub. No.: US 2020/0107563 A1
(43) Pub. Date: Apr. 9, 2020

(54) MOLECULAR MODIFIER FOR COMBUSTIBLE AND NON-COMBUSTIBLE FLUIDS, LIQUIDS AND GASES

(71) Applicant: Adrian Ernesto Alvarez Lhabriel, Guayaquil (EC)

(72) Inventor: Adrian Ernesto Alvarez Lhabriel, Guayaquil (EC)

(21) Appl. No.: 16/539,779

(22) Filed: Aug. 13, 2019

Related U.S. Application Data
(63) Continuation-in-part of application No. PCT/IB2017/050797, filed on Feb. 13, 2017.

(51) Int. Cl. A23L 5/30; A23L 3/32; A23L 2/50; A61M 16/10; A61G 10/02

(52) U.S. Cl. CPC A23L 5/30; A23L 3/32; A23V 2002/00; A61M 16/1005; A61G 10/026; A23L 2/50

(57) ABSTRACT

The present magnetic and electromagnetic equipment achieves the molecular modification of liquid and gaseous fluids combustible or non-combustible by combining magnetic turbulence. The device combines magnetic and electromagnetic fields for producing magnetic turbulence that modifies the atomic behavior diamagnetic materials to be processed by the covalence thereof, improving the assimilation of fluids in humans by increasing the surfactant quality of the fluids in physical states.

### CROSS-REFERENCE TO RELATED APPLICATION

The present application claims priority as a continuation-in-part application from International Application Serial Number PCT/162017/050797 filed on Feb. 13, 2017, which is incorporated herein by reference in its entirety.

### FIELD OF INVENTION

The object of this invention is to modify the molecular structure of fluids; (liquid and gaseous) in all their variable classification; hence, combustible and non-combustible, alcoholic or non-alcoholic, whether these are potable or non-potable. The fluids pass through a temporary treatment of altered states of energy that excites its peripheral electrons, generating clashes between them that may be excite by a later state of energy, using an equipment made of magnetic and electromagnetic fields. This leads to the stereo-chemical deformation of its molecular components enhancement and improvement of its organoleptic characteristics, fuels and exploitation of its use, with a continuous and stable method in an expeditious manner, optimizing energy production costs in its operation.

### BACKGROUND

In traditional designs for the exploitation of liquid or gaseous fluids in the fields of food or medical industry, fixated magnetic fields in combinations with ultrasound and mechanical turbulence systems are use, such as atomization by pressurized cavitation chambers to achieve modular production systems, limited in production volumes. The published North American application patent No 2003/0110951 proposes a beverage maturation process by ultrasound with a power of 5 W/L between 70-150° F. The Japanese patent 356068385 proposes accelerating the maturation of beverages with 16-60 kHz of ultrasonic radiation, promoting a chemical reaction causing condensation, oxidation, and polymerization, among others. This result in differentiation on taste in the mouth, smell and body and the U.S. Pat. No. 5,173,318 includes the introduction of Ozone in cold beverages by applying vibrations of a frequency between 15-150 kHz to vibrate and heat the fermented product above 35° C. to oxidize the fermented product.

The effect of sonic cavitation decreases with the increase in distance of the course of the radiation depending on the vessel's size, its frequency, and the length of its wave, which makes it less efficient, so they do not offer, nor optimize the objective because of its high-energy cost.

As well, other proposed techniques for the treatment of liquid fluids such as the U.S. Pat. No. 6,063,267A that uses one or more external coils to the equipment to take advantage of the electromagnetism with the objective of reducing or getting rid of microorganisms, and dissolved solids to avoid clogging in pipes, equipment and other mechanisms where the treated fluid passes.

The US2016016829A1 uses magnetics fields induced by a power source, which is not magnetism but its electromagnetism, and two types of submergible and variable electromagnetic coils that get in contact with the liquid to get rid of calcium carbonate ions.

The utility model ES1176659U proposes the use of a group of six magnets hexagonally ensemble around an interiorly notched pipe next to an inductor coil for the treatment of the fluids used in ornamental and agricultural production plants, since it reduces the amount of solved minerals in irrigation water.

### DESCRIPTION OF THE DRAWINGS

These features and advantages of described in this application will become apparent from the detailed description of the following embodiments in conjunction with the accompanying drawings, wherein:

FIG. 1 is a plan view of one embodiment.

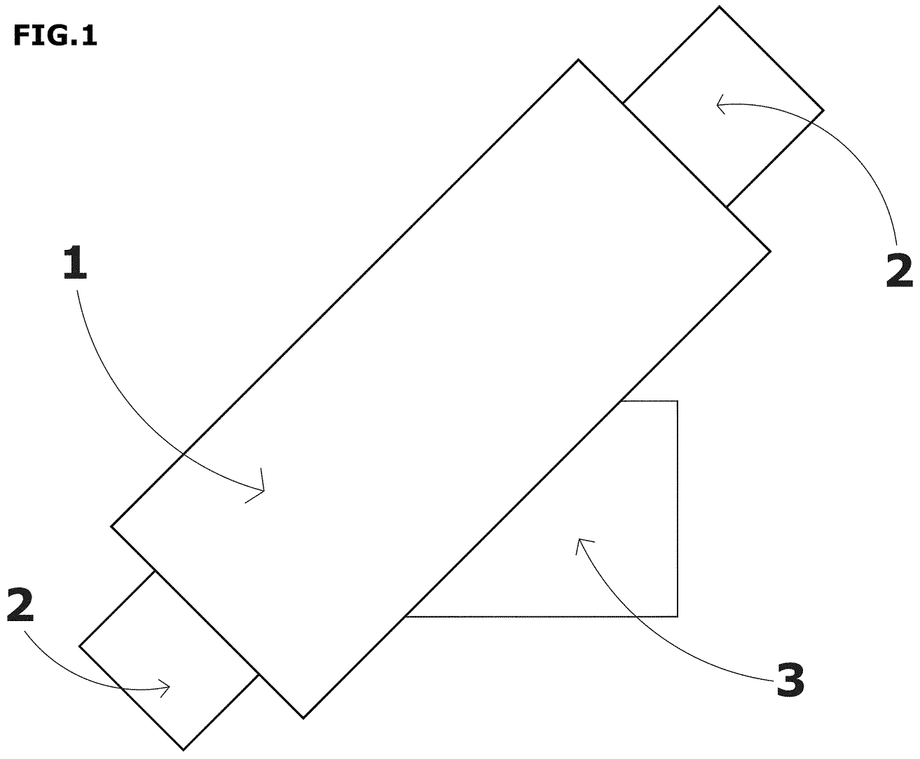

FIG. 2 is a plan view of one embodiment.

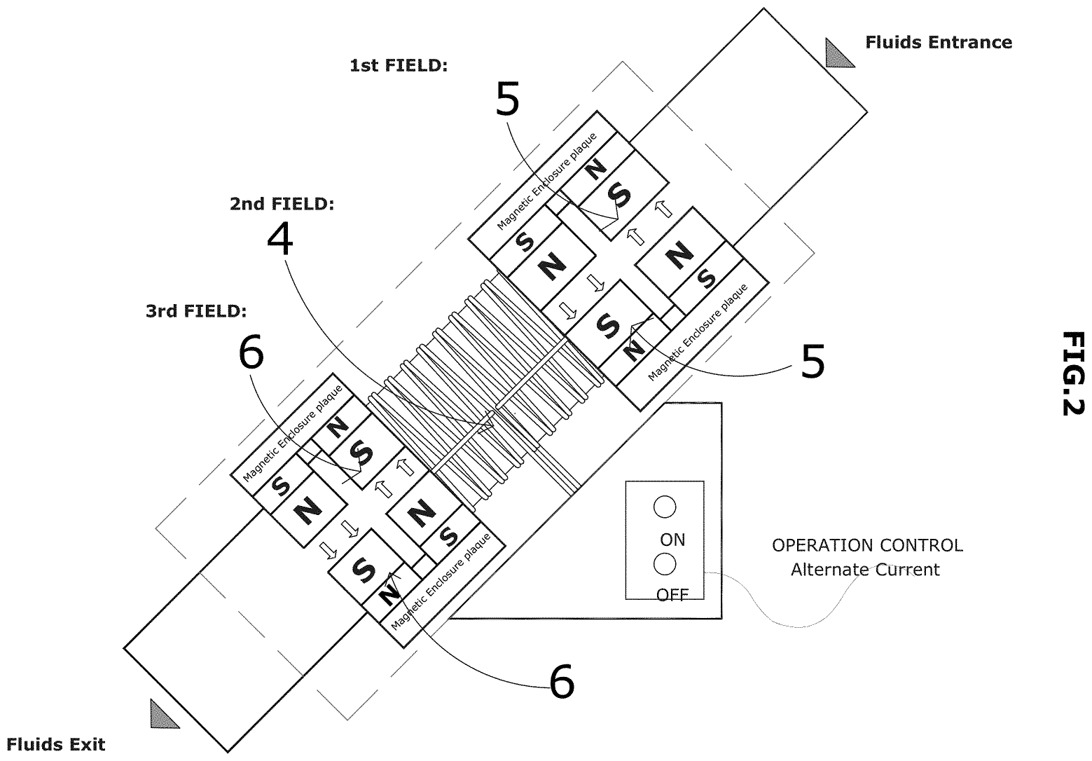

### DESCRIPTION OF THE INVENTION

The present invention consists of two bodies that work together by hermetic, very resistant seal/joint. The first body consists of two non-ferrous metal tubes, one outer tube with a larger diameter and the other with a smaller diameter, between which the magnetic and electromagnetic elements are place. The second body consists of a metal case made to host the electrical control system of operation.

Between the tubes of the first body, centered, a coil will be place between 300 to 1000 turns of 1.5 to 3 mm in diameter, powered by a frequency of 100 to 600 Hz, to create an axial magnetic field. At its ends two magnetic systems are placed formed by four fixed rectangular magnets with two magnetic enclosure plaques generating a field of closed domain bidirectional uni-positional not less than 5000 Gauss each one creating several magnetic fields transversal to the inner tube and orthogonal to the magnetic field of the high frequency coil. A voltage of 0.5 to 2 Volts will be applied to the coil, in such a way that an intensity of approximately 50 to 300 Amps circulates through it, creating a third magnetic field orthogonal to the first two.

The combination of orthogonal fixed magnetic fields, with the combination of electromagnetic fields that can be either: linear, axial, or fixed flow; in relation to fixed magnetic fields with the obtaining of magnetic turbulences that achieve oscillating fields that imply the stereo-chemical deformation of its molecular components. This generates molecularly improved fluids; allow a volumetric production of these, without the combination of any mechanical system.

The present invention based on Faraday's law, on the Hall Effect, and on the application of the magneto hydrodynamic produced by the moment in which the fluids circulate through the interior pipe. Using the effect of the vertiginous electric fields at atomic and molecular level through the induction produced by magnetic turbulence fields, being able to vary on the application of the electromagnetic fields on its electric part. These coils can be powered up with alternating or continuous current; single-phase or three-phase, high or low intensity and frequency, as well as the magnetic elements that form the orthogonal fields that can be ceramic magnets, rare earths, Neodymium etc. Consider that the minimum values of the magnetic fields to achieve this effect are greater than 5000 Gauss/cm per gram molecule, all this depending on the type of fluid.

All the magnetic as electromagnetic elements will be mount on a pipe made of paramagnetic material, which must have a thermos-insulating coating. The fix magnets will be surround by a magnetic conductor of high performance, to concentrate and direction of the magnetic fields (depending on the production process).

Both the magnetic as electromagnetic components will be attach with expansive foam or resin, depending on the work for which the equipment is needed, and the total sealed assembly will be armored with a magnetic mechanical protector that prevents the filtration of magnetism to contiguous equipment that can be affected by these, optimizing its operation.

For production, a single equipment design as per the working flow to be modify to improve its properties is enough. The space require for its use is relatively small, from 0.60 to 1.00 meters long.

The present process modifies the molecules of liquid fluids affecting their covalence, ionizing chemical components and revitalizing organic components intensifying their natural properties for a long period and in a minimum of time.

This closed treatment system can be applied in hot or cold beverages such as coffee, chocolate, tea, natural or artificial juices, water, alcoholic beverages, blood; and gases such as medicinal oxygen, for combination or mixture of different types of gaseous fluids, as well as in hyperbaric systems, among other uses.

The stability of the obtained product has been demonstrated in retests carried out by tasters allowing more than 90 days to go back to test it, checking its effectiveness over time because after being treated by this system, the molecule does not return to its previous state.

In a further embodiment, a closed treatment system is applied in hot or cold beverages such as coffee, chocolate, tea, natural or artificial juices, water, alcoholic beverages, blood; and gases such as medicinal oxygen, for combination or mixture of different types of gaseous fluids, as well as in hyperbaric systems.

What is claimed is:

1. A device for modification of liquid or gaseous fluids characterized by the application of magnetic and electromagnetic fields to modify the molecular structure of liquid and gaseous fluids, combustible and non-combustible, alcoholic or non-alcoholic, whether these are potable or non-potable for the potentiation and improvement of its organoleptic characteristics, combustible characteristics, and use, with a continuous and stable method.

2. The device of claim 1, composed internally by magnetic and electromagnetic parts to achieve the molecular modification of liquid fluids and gases, combustible and non-combustible, alcoholic or non-alcoholic, whether these are potable or non-potable.

3. The device of claim 1, made of metallic material and consists of two bodies.

4. The device of claim 3, formed by two tubes of non-ferrous material and consists of two pipes, one outer tube of larger diameter, and one inner tube of smaller diameter.

5. The device of claim 3 consists of a metal casing welded to the outer pipe of the first body.

6. The device of claim 4, wherein the inner tube is center of a coil of between 300 to 1000 turns of 1.5 to 3 mm in diameter, fed by a frequency of 100 to 600 Hz.

7. The device of claim 6, wherein the coil creates an axial magnetic field, in relation to the fluids circulating in the inner tube.

8. The device of claim 6, wherein the ends of the coil alternate and include circular and rectangular magnets having a closed domain bidirectional uni-positional of not less than 5000 Gauss each.

9. The device of claim 8, wherein the magnets create several magnetic fields transverse to the inner tube and orthogonal to the magnetic field of the coil, of medium frequency.

10. The device of claim 6, wherein a tension of 0.5 V or more is applied to the coil, so that an intensity of 50 to 300 Amps circulates through it.

11. The device of claim 10, wherein the tension applied to the coil creates a third magnetic field orthogonal to the two fixed magnets, obtaining magnetic turbulences.

12. The device of claim 1, wherein the device can change its external shape, electric, dimensions, shapes, and potency of its internal components, according to the specific applications of the use of magnetic and electromagnetic fields.

13. The device of claim 1, based on Faraday's law, on the Hall Effect, and on the application of the magneto hydro dynamic produced by the moment in which the fluids circulate through the inner pipe taking advantage of the effect produced by vertiginous electric fields produced at atomic and molecular level through the induction produced by the fields of magnetic turbulence.

14. The device of claim 8, wherein the magnetic elements that form the orthogonal fields can be ceramic magnets, rare earth, Neodymium, or other materials.

15. The device of claim 8, wherein the magnetic and electromagnetic elements are mounted on a pipe of paramagnetic material.

16. A process for modifying molecules of liquid fluids, affecting their covalence, ionizing chemical components and revitalizing organic components intensifying their natural properties for a long period and in a minimum of time.

17. A device comprising three magnetic fields having a premeditated position, wherein a first field is produced by a system of mid potency fixed magnets, a second field is produced by an electromagnetic coil of high frequency and high amperage, and a third field produced by a system of fixed magnets of greater potency than the first field but lower than the second field.
